(19) United States
(12) Patent Application Publication
(10) Pub. No.: US 2009/0081742 A1
(43) Pub. Date: Mar. 26, 2009

(54) HIGH EFFICIENCY SEPARATIONS TO RECOVER OIL FROM MICROALGAE

(76) Inventors: Eric H. Dunlop, Paradise (AU); David A. Hazlebeck, El Cajon, CA (US)

Correspondence Address: NYDEGGER & ASSOCIATES, 348 OLIVE STREET, SAN DIEGO, CA, 92103, US

(21) Appl. No.: 11/860,327

(22) Filed: Sep. 24, 2007

(51) Int. Cl. C12P 7/02; C12M 1/00

(52) U.S. Cl. 435/155; 435/289.1

(57) ABSTRACT

A system and method for processing algae cells to create biofuel are disclosed. Specifically, the system and method utilize steam to rupture algae cells in order to utilize intracellular oil therein. The system includes a conduit for growing algae cells and a generator for creating steam. Further, the system includes a lysing device that mixes the algae cells and the steam to rupture the algae cells. In order to maximize the efficiency of the lysing process, the system may further include a heat exchanger for preheating the algae cells with the lysed cells. In addition, the system includes a bioreactor to synthesize biofuel from the unbound oil.

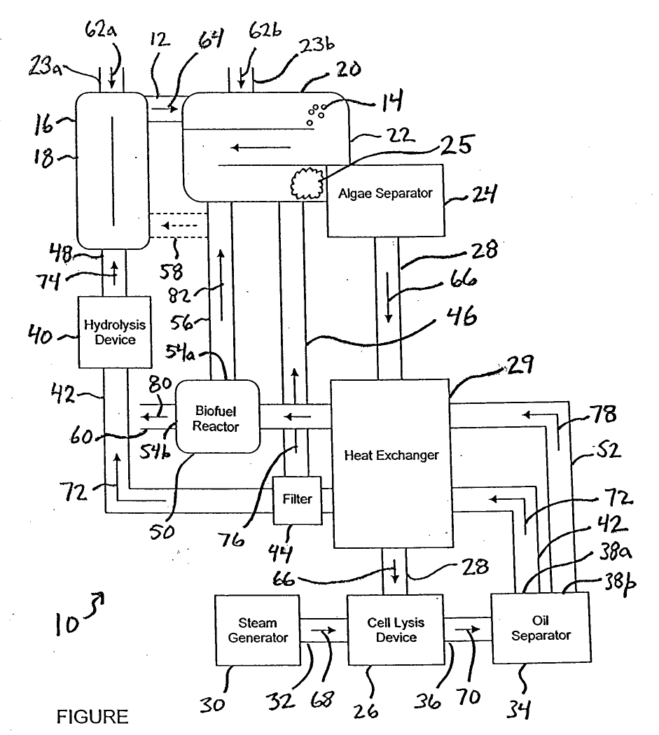

### FIELD OF THE INVENTION

The present invention pertains generally to processes for separating intracellular materials from one another. More particularly, the present invention pertains to a lysing system and method for rupturing cells to unbind intracellular material. The present invention is particularly, but not exclusively, useful as a system and method for separating intracellular oil from other cell matter in algae for use in the creation of biofuel from the intracellular oil.

### BACKGROUND OF THE INVENTION

As worldwide petroleum deposits decrease, there is rising concern over shortages and the costs that are associated with the production of hydrocarbon products. As a result, alternatives to products that are currently processed from petroleum are being investigated. In this effort, biofuel such as biodiesel has been identified as a possible alternative to petroleum-based transportation fuels. In general, a biodiesel is a fuel comprised of mono-alkyl esters of long chain fatty acids derived from plant oils or animal fats. In industrial practice, biodiesel is created when plant oils or animal fats are reacted with an alcohol, such as methanol.

For plant-derived biofuel, solar energy is first transformed into chemical energy through photosynthesis. The chemical energy is then refined into a usable fuel. Currently, the process involved in creating biofuel from plant oils is expensive relative to the process of extracting and refining petroleum. It is possible, however, that the cost of processing a plant-derived biofuel could be reduced by minimizing the costs associated with extracting plant oils. Because algae is known to be one of the most efficient plants for converting solar energy into cell growth, it is of particular interest as a biofuel source. However, current algae processing methods have failed to result in a cost effective algae-derived biofuel.

In overview, the biochemical process of photosynthesis provides algae with the ability to convert solar energy into chemical energy. During cell growth, this chemical energy is used to drive synthetic reactions, such as the formation of sugars or the fixation of nitrogen into amino acids for protein synthesis. Excess chemical energy is stored in the form of fats and oils as triglycerides. Thus, the creation of oil in algae only requires sunlight, carbon dioxide and the nutrients necessary for formation of triglycerides. However, the extraction of triglycerides from algae is typically not efficient and the associated costs are high.

In light of the above, it is an object of the present invention to provide a system and method for processing oil from algae which reduces processing costs. For this purpose, a number of systems have been developed, such as those disclosed in co-pending U.S. patent application Ser. No. ______ for an invention entitled “Transportable Algae Biodiesel System,” which is filed concurrently herewith, co-pending U.S. patent application Ser. No. 11/549,532 for an invention entitled “Photosynthetic Oil Production in a Two-Stage Reactor” filed Oct. 13, 2006, co-pending U.S. patent application Ser. No. 11/549,541 for an invention entitled “Photosynthetic Carbon Dioxide Sequestration and Pollution Abatement” filed Oct. 13, 2006, co-pending U.S. patent application Ser. No. 11/549,552 for an invention entitled “High Photoefficiency Microalgae Bioreactors” filed Oct. 13, 2006, and co-pending U.S. patent application Ser. No. 11/549,561 for an invention entitled “Photosynthetic Oil Production with High Carbon Dioxide Utilization” filed Oct. 13, 2006. All aforementioned co-pending U.S. patent applications are assigned to the same assignee as the present invention, and are hereby incorporated by reference. Another object of the present invention is to provide a system for efficiently separating intracellular materials in algae cells. Still another object of the present invention is to provide a system for harvesting oil from algae. Another object of the present invention is to provide a system for lysing algae cells to unbind intracellular oil. Another object of the present invention is to provide a system for processing oil from algae that utilizes live steam to rupture algae cells. Yet another object of the present invention is to provide a system and method for processing algae with high oil content that is simple to implement, easy to use, and comparatively cost effective.

### SUMMARY OF THE INVENTION

In accordance with the present invention, a system and method are provided for the creation of biofuel from oil in algae. In the system and method, algae cells are lysed to efficiently process the cells' intracellular oil. For this purpose, the system utilizes steam to rupture algae cells and to unbind the intracellular oil. Structurally, the system includes a chemostat that defines a conduit for growing algae cells. Further, the system includes a plug flow reactor that defines a conduit for fostering oil production within the algae cells. For the present invention, the plug flow reactor is positioned to receive material from the chemostat.

In addition to the chemostat and plug flow reactor, the system includes an algae separator. Specifically, the algae separator is positioned in fluid communication with the plug flow reactor to remove the algae cells from the plug flow reactor's conduit. Further, the system includes a generator for creating steam. Also, the system includes a device for lysing algae cells to unbind oil from the algae cells. Specifically, the lysing device mixes live steam from the generator with the algae cells to rupture the cells. For this purpose, the lysing device is positioned to receive algae cells from the algae separator.

For purposes of the present invention, the system also includes a heat exchanger for transferring heat between the heated outputs and the non-heated inputs of the lysis device. Specifically, the heat exchanger transfers heat from lysed cell material to algae cells that have not yet entered the lysis device. In this manner, heating costs are reduced. Also, the system includes a bioreactor for synthesizing biofuel from the unbound oil.

In operation, algae cells are grown in the chemostat and are continuously transferred to the plug flow reactor. In the plug flow reactor, the rate of intracellular oil production in the algae cells is increased. After the algae cells have attained a high oil content, the algae separator concentrates the algae cells for removal from the plug flow reactor and delivers them to the cell lysis device through a pipe that passes through the heat exchanger. Then, the cell lysis device mixes live steam with the cells to rupture the cells and unbind the intracellular oil from the remaining cell matter. This unbound cell material is passed through the heat exchanger in order to preheat the incoming algae cells. Thereafter, the unbound intracellular oil is synthesized into biofuel by the bioreactor.

### BRIEF DESCRIPTION OF THE DRAWINGS

The novel features of this invention, as well as the invention itself, both as to its structure and its operation, will be best understood from the accompanying drawing, taken in conjunction with the accompanying description, in which the FIGURE is a schematic view of the system for lysing algae cells in accordance with the present invention.

### DESCRIPTION OF THE PREFERRED EMBODIMENTS

Referring to the FIGURE, a system for lysing algae cells in accordance with the present invention is shown and generally designated 10. Specifically, in the system 10, steam is used to efficiently lyse algae cells to facilitate the use of intracellular oil. As shown, the system 10 includes a conduit 12 for growing algae cells 14 with high oil content. As further shown, the conduit 12 includes an upstream conduit section 16 that is defined by a continuously stirred first stage reactor or chemostat 18. Also, the conduit 12 includes a downstream conduit section 20 that is defined by a plug flow second stage reactor 22. In this manner, the conduit 12 passes through the chemostat 18 and the plug flow reactor 22. For purposes of the present invention, the conduit 12 is provided with ports 23a and 23b for receiving input materials into the upstream conduit section 16 and the downstream conduit section 20, respectively.

As further shown in the FIGURE, the system 10 includes an algae separator 24 that is in fluid communication with the downstream conduit section 20 in the plug flow reactor 22. For purposes of the present invention, the algae cells 14 are concentrated in the downstream conduit section 20 to form an algae cell concentrate 25. Further, the algae separator 24 removes the algae cell concentrate 25 from the downstream conduit section 20. Also, the system 10 includes a cell lysis device 26 that receives algae cell concentrate 25 from the algae separator 24 via pipe 28. In the present invention, the pipe 28 passes through a heat exchanger 29 for preheating as is more fully explained below.

As shown, the cell lysis device 26 is connected in fluid communication with a steam generator 30 via a pipe 32. Also, the cell lysis device 26 is shown to be in fluid communication with an oil separator 34. Specifically, a pipe 36 interconnects the cell lysis device 26 and the oil separator 34. For purposes of the present invention, the oil separator 34 is provided with two outlets 38a-b. As shown, the outlet 38a is connected to a hydrolysis device 40 by a pipe 42 that passes through a filter 44. Also, the pipe 42 passes through the heat exchanger 29 to transfer heat to the pipe 28. For the present invention, the filter 44 is connected directly to the downstream conduit section 20 by a pipe 46. Further, the hydrolysis device 40 is connected to the upstream conduit section 16 of the chemostat 18 by a pipe 48.

Referring back to the oil separator 34, it can be seen that the outlet 38b is connected to a biofuel reactor 50 by a pipe 52 that passes through the heat exchanger 29 to transfer heat to the pipe 28. It is further shown that the biofuel reactor 50 includes two exits 54a-b. For purposes of the present invention, the exit 54a is connected to the downstream conduit section 20 of the plug flow reactor 22 by a pipe 56. Additionally or alternatively, the exit 54a may be connected to the upstream conduit section 16 of the chemostat 18 by a pipe 58. As further shown, the exit 54b is connected to a pipe 60 which may connect to a tank or reservoir (not shown) for purposes of the present invention.

In operation of the present invention, algae cells 14 are initially grown in the upstream conduit section 16 in the chemostat 18. Specifically, a medium with a nutrient mix 62a is continuously fed into the upstream conduit section 16 through the port 23a at a selected rate. Further, the conditions in the upstream conduit section 16 are maintained for maximum algal growth. For instance, in order to maintain the desired conditions, the medium 62a and the algae cells 14 are moved around the upstream conduit section 16 at a preferred fluid flow velocity of approximately fifty centimeters per second. Further, the amount of algae cells 14 in the upstream conduit section 16 is kept substantially constant. Specifically, the medium with nutrient mix 62a is continuously fed into the upstream conduit section 16 through the port 23a and an effluence 64 containing algae cells 14 is continuously removed from the upstream conduit section 16 as overflow. Under preferred conditions, approximately one to ten grams of algae per liter of fluid circulate in the upstream conduit section 16. Preferably, the residence time for algae cells 14 in the upstream conduit section 16 is about one to five days.

After entering the downstream conduit section 20, the effluence 64 containing algae cells 14 moves in a plug flow regime. Preferably, the effluence 64 moves through the downstream conduit section 20 of the plug flow reactor 22 at a rate of between ten and one hundred centimeters per second. Further, as the effluence 64 moves downstream, a modified nutrient mix 62b may be added to the downstream conduit section 20 through the port 23b. This modified nutrient mix 62b may contain a limited amount of a selected constituent, such as nitrogen or phosphorous. Alternatively, no further material may be added through the port 23b and selected constituents in the effluence 64 may be exhausted. The absence or small amount of the selected constituent causes the algae cells 14 to focus on energy storage rather than growth. As a result, the algae cells 14 form triglycerides.

At the end of the downstream conduit section 20, the algae cells 14 form the algae cell concentrate 25 that the algae separator 24 removes from the effluence 64. To facilitate this process, the depth of the downstream conduit section 20 may be increased near the algae separator 24. The corresponding increase in the fluid flow cross-sectional area, and decrease in fluid flow rate, allows the algae cells 14 to settle to the bottom of the conduit section 20 forming the algae cell concentrate 25. In certain embodiments, the modified nutrient mix 62b may include a limited amount of a predetermined constituent to trigger flocculation of the algae cells 14 in the downstream conduit section 20. The predetermined constituent may be the same as the selected constituent such that a shortage of nitrogen, for example, causes both the production of triglycerides and the flocculation of the algae cells 14 to form the concentrate 25.

After the algae cell concentrate 25 is removed from the conduit 12 by the algae separator 24, it is delivered to the cell lysis device 26. As shown, the algae cell concentrate 25 passes through the pipe 28 (and through the heat exchanger 29) to the cell lysis device 26 as indicated by arrows 66. For purposes of the present invention, the cell lysis device 26 lyses the algae cells 14 in the algae cell concentrate 25 to unbind the oil therein from the remaining cell matter. Specifically, steam (identified by arrow 68) created by the steam generator 30 is delivered to the lysis device 26 through pipe 32. Inside the lysis device 26, the live steam 68 is directly mixed with the algae cell concentrate 25 causing cell lysis and an increase in temperature and water content of the (now ruptured) algae cells 14 within the concentrate 25. Preferably, the amount of steam utilized is between about 2-20% of the mass of the incoming algae cell concentrate 25, and most preferably about 5%. In other words, the mass flow rate of the steam M_{S} is approximately 2-20%, and more preferably approximately 2-5% of the mass flow rate of the algae cell concentrate M_{A}. Further, the steam 68 preferably is at a pressure of about 3-5 bar.

After the lysing process occurs, the unbound oil and remaining cell matter, collectively identified by arrow 70, are passed through pipe 36 to the oil separator 34. Thereafter, the oil separator 34 withdraws the oil from the remaining cell matter as is known in the art. After this separation is performed, the oil separator 34 discharges the remaining cell matter (identified by arrow 72) out of the outlet 38a and through the pipe 42, with the remaining cell matter 72 eventually reaching the chemostat 18. As shown, the remaining cell matter 72 passes through the heat exchanger 29 in order to transfer heat to the algae cell concentrate 66 in the pipe 28.

In the chemostat 18, the remaining cell matter 72 is utilized as a source of nutrients and energy for the growth of algae cells 14. Because small units of the remaining cell matter 72 are more easily absorbed or otherwise processed by the growing algae cells 14, the remaining cell matter 72 may first be broken down before being fed into the chemostat 18. To this end, the hydrolysis device 40 is interconnected between the oil separator 34 and the chemostat 18. Accordingly, the hydrolysis device 40 receives the remaining cell matter 72 from the oil separator 34, hydrolyzes the received cell matter 72, and then passes hydrolyzed cell matter (identified by arrow 74) to the chemostat 18 through the pipe 48. Alternatively, large units 76 of the remaining cell matter 72 may be removed from the pipe 42 by the filter 44. These large units 76 of cell matter 72 are delivered to the downstream conduit section 20 through the pipe 46 to be used as a flocculation aid.

Referring back to the oil separator 34, it is recalled that the remaining cell matter 72 was discharged through the outlet 38a. At the same time, the oil withdrawn by the oil separator 34 is discharged through the outlet 38b. Specifically, the oil (identified by arrow 78) is delivered to the biofuel reactor 50 through the pipe 52. In order to efficiently utilize the energy contained in the heated oil 78, the oil 78 passes through the heat exchanger 29 and transfers heat to the algae cells 66 in the pipe 28. In the biofuel reactor 50, the oil 78 is reacted with alcohol, such as methanol, to create mono-alkyl esters, i.e., biodiesel. This biodiesel (identified by arrow 80) is released from the exit 54b of the biofuel reactor 50 through the pipe 60 to a tank, reservoir, or pipeline (not shown) for use as fuel. In addition to the biodiesel 80, the reaction between the oil 78 and the alcohol produces glycerin as a byproduct. For purposes of the present invention, the glycerin (identified by arrow 82) is pumped out of the exit 54a of the biofuel reactor 50 through the pipe 56 to the plug flow reactor 22.

In the plug flow reactor 22, the glycerin 82 is utilized as a source of carbon by the algae cells 14. Importantly, the glycerin 82 does not provide any nutrients that are otherwise being kept at a limited amount to induce oil production by the algae cells 14 or to trigger flocculation. Preferably, the glycerin 82 is added to the plug flow reactor 22 at night to aid in night-time oil production. Further, because glycerin 82 would otherwise provide bacteria and/or other non-photosynthetic organisms with an energy source, limiting the addition of glycerin 82 to the plug flow reactor 22 only at night allows the algae cells 14 to utilize the glycerin 82 without facilitating the growth of foreign organisms. As shown in the FIGURE, the exit 54a of the biofuel reactor 50 may also be in fluid communication with the chemostat 18 via the pipe 58 (shown in phantom). This arrangement allows the glycerin 82 to be provided to the chemostat 18 as a carbon source.

As discussed above, the heat exchanger 29 provides for the transfer of heat between the heated outputs and the non-heated inputs of the lysis device 26. As shown, the algae cell concentrate 25 flows from the algae separator 24 to the lysis device 26 through the pipe 28 which passes through the heat exchanger 29. Typically, the algae cell concentrate 25 enters the heat exchanger 29 at a temperature of about 20° C. At the same time, lysed cells in the form of unbound oil and remaining cell matter 70 flow through the heat exchanger 29. Specifically, the remaining cell matter 72 and the oil 78 flow through the heat exchanger 29 in pipes 42 and 52, respectively. Preferably, the remaining cell matter 72 and oil 78 have a temperature of about 100° C. upon entering the heat exchanger 29. After heat is transferred between the pipes 42 and 52 and the pipe 28, the algae cell concentrate 25 exits the heat exchanger 29 at a temperature of about 80° C., while the remaining cell matter 72 and oil 78 exit the heat exchanger 29 at a temperature of about 40° C. While the FIGURE illustrates a system 10 in which the remaining cell matter 72 and oil 78 are separated before passing through the heat exchanger 29, it is contemplated that the heat exchange could be performed before the oil separation process. However, it is noted that separation before cooling can reduce the tendency for the formation of an emulsion in the unbound oil and remaining cell matter 70.

While the particular High Efficiency Separations to Recover Oil From Microalgae as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for processing oil from algae to create biofuel which comprises:
a conduit for growing algae cells with an oil content;
an algae separator in fluid communication with the conduit for receiving an effluent with algae cells, and for removing an algae cell concentrate therefrom;
a device for lysing the algae cells with steam, said device receiving the algae cell concentrate removed from the algae separator, with said steam causing the algae cells in the algae cell concentrate to rupture to unbind oil therein; and
a bioreactor for synthesizing biofuel from the unbound oil, said bioreactor receiving the oil from the lysing device.

2. A system as recited in claim 1 further comprising a steam generator for supplying steam to the lysing device, wherein the algae cell concentrate has a mass flow rate of M_{A} and the steam has a mass flow rate of M_{S}, with M_{S} being equal to approximately 2-20% of M_{A}.

3. A system as recited in claim 1 further comprising a heat exchanger for preheating the algae cell concentrate before lysing, with said heat exchanger receiving lysed cells from the lysing device, receiving the algae cell concentrate removed from the algae separator, and transferring heat from the lysed cells to the algae cell concentrate removed from the algae separator.

4. A system as recited in claim 3 wherein the algae cell concentrate is preheated to between about 40-90° C.

5. A system as recited in claim 3 further comprising an oil separator for receiving the lysed cells from the lysis device and for separating oil from remaining cell matter in the lysed cells, with said oil separator being interconnected between the lysis device and the bioreactor.

6. A system as recited in claim 5 wherein the oil separator separates the oil and the remaining cell matter in the lysed cells before the lysed cells are delivered to the heat exchanger.

7. A system as recited in claim 5 wherein said oil separator is in fluid communication with the conduit for recycling the remaining cell matter to the conduit to support growth of algae cells.

8. A system for processing oil from algae to create biofuel which comprises:
a conduit for flowing an effluent including algae cells;
an algae separator in fluid communication with the conduit for removing an algae cell concentrate therefrom;
a generator for creating steam;
a device for lysing the algae cells, said device receiving the algae cell concentrate from the algae separator and the steam from the generator, with said steam causing the algae cells to rupture to unbind oil therein; and
a bioreactor for synthesizing biofuel from the unbound oil, said bioreactor receiving the oil from the lysing device.

9. A system as recited in claim 8 wherein the algae cells have a mass flow rate of M_{A} and the steam has a mass flow rate of M_{S}, with M_{S} being equal to approximately 2-20% of M_{A}.

10. A system as recited in claim 8 further comprising a heat exchanger for preheating the algae cell concentrate before lysing, with said heat exchanger receiving lysed cells from the lysing device, receiving the algae cell concentrate from the algae separator, and transferring heat from the lysed cells to the algae cell concentrate from the algae separator.

11. A system as recited in claim 10 wherein the algae cell concentrate is preheated to between about 40-90° C.

12. A system as recited in claim 11 wherein the algae cell concentrate is preheated from about 20° C. to about 80° C. by the heat exchanger and wherein the lysed cells are cooled from about 100° C. to about 40° C. by the heat exchanger.

13. A system as recited in claim 10 further comprising an oil separator for receiving the lysed cells from the lysis device and for separating oil from remaining cell matter in the lysed cells, with said oil separator being interconnected between the lysis device and the bioreactor.

14. A system as recited in claim 13 wherein the oil separator separates the oil and the remaining cell matter in the lysed cells before the lysed cells are delivered to the heat exchanger.

15. A system as recited in claim 14 wherein said oil separator is in fluid communication with the conduit for recycling the remaining cell matter to the conduit to support growth of algae cells.

16. A method for processing oil from algae to create biofuel which comprises the steps of:
flowing an effluent including algae cells through a conduit;
removing an algae cell concentrate from the effluent;
creating steam;
mixing the algae cell concentrate and the steam, with the steam causing the algae cells to rupture to unbind oil therein; and
synthesizing biofuel from the unbound oil.

17. A method as recited in claim 16 wherein during the mixing step, the algae cell concentrate has a mass flow rate of M_{A} and the steam has a mass flow rate of M_{S}, with M_{S} being equal to approximately 2-20% of M_{A}.

18. A method as recited in claim 16 further comprising the step of preheating algae cell concentrate removed from the effluent before lysing with previously lysed cells.

19. A method as recited in claim 18 further comprising the step of separating oil from remaining cell matter in the lysed cells.

20. A method as recited in claim 19 wherein the separating step is performed before the preheating step.

21. A method for processing oil from algae to create biofuel which comprises the steps of:
flowing an effluent including algae cells through a conduit;
flocculating the algae cells to form an algae cell concentrate;
removing the algae cell concentrate from the effluent;
lysing algae cells in the algae cell concentrate to create unbound oil and intracellular material;
separating a portion of the intracellular material and using the separated portion to aid in the flocculating step; and
synthesizing biofuel from the unbound oil.

22. A method as recited in claim 21 wherein the intracellular material used in the flocculating step contains DNA.

23. A method as recited in claim 21 wherein the intracellular material used in the flocculating step contains polysaccharide.
